(19) United States
(12) Patent Application Publication
(10) Pub. No.: US 2015/0359432 A1
(43) Pub. Date: Dec. 17, 2015

(54) METHODS AND DEVICES FOR DETECTING NERVE ACTIVITY

(71) Applicant: Sympara Medical, Inc., San Mateo, CA (US)

(72) Inventors: Kevin Joe Ehrenreich, San Francisco, CA (US); Randolf von Oepen, Aptos, CA (US); William Rehlich, San Mateo, CA (US); Kelly Justin McCrystle, Menlo Park, CA (US); Eric Beuhlmann, San Mateo, CA (US); Niel Barman, San Mateo, CA (US)

(21) Appl. No.: 14/736,049

(22) Filed: Jun. 10, 2015

Related U.S. Application Data
(60) Provisional application No. 62/010,960, filed on Jun. 11, 2014.

(51) Int. Cl. A61B 5/00; A61B 8/00; A61B 8/12; A61B 5/04; A61B 18/14

(52) U.S. Cl. CPC A61B 5/0051; A61B 5/6853; A61B 5/04001; A61B 5/4836; A61B 18/1492; A61B 8/12; A61B 8/4483; A61B 2018/00577

(57) ABSTRACT

Devices, systems and methods are described which are capable of producing vibrational energy within a lumen of a mammal and further including a measurement sensor capable of detecting, measuring and recording nerve activity in response to the vibrational energy.

### CROSS-REFERENCE TO RELATED APPLICATIONS

This application claims the benefit as a nonprovisional application of U.S. Provisional Application No. 62/010,960, filed Jun. 11, 2014, the disclosure of which is incorporated by reference herein in its entirety. Any and all applications for which a foreign or domestic priority claim is identified in the Application Data Sheet as filed with the present application are hereby incorporated by reference under 37 CFR 1.57.

### BACKGROUND

This disclosure relates generally to methods and devices for the detection and/or activation of nerve activity within a lumen of a mammal. More specifically, methods and devices to be utilized before, during or after a denervation procedure are disclosed.

Hypertension, or high blood pressure, affects millions of people every day and is a serious health hazard. Hypertension is associated with an elevated risk for heart attack, heart failure, arterial aneurysms, kidney failure and stroke. There are many factors that may affect blood pressure, such as: salt intake, obesity, occupation, alcohol intake, smoking, pregnancy, stimulant intake, sleep apnea, genetic susceptibility, decreased kidney perfusion, arterial hardening and medication(s). Many times people are unaware that they suffer from hypertension until it is discovered during a medical check-up with their health care practitioner (HCP), or worse, it is discovered when they are hospitalized for a hypertension related condition such as a heart attack or stroke.

As stated above, hypertension currently affects a large and growing population. Currently treatments for hypertension range from prescribed lifestyle changes to the use of pharmaceutical products. Within the past couple of years, new surgical therapies are emerging. These surgical therapies may lead to the implantation of a device for stimulating a patient's carotid baroreceptor.

Another type of medical device being developed is a catheter based system, wherein the catheter includes electrodes, the catheter is advanced within the renal artery, wherein the electrodes are utilized to burn or otherwise disconnect a portion of the nerves of the kidney. This surgical procedure is commonly referred to as renal denervation. Many companies are developing renal denervation devices. The devices may be disposed internally to the artery or external to the artery/patient, and they utilize many different methods to undertake the denervation. For example, electrical energy may be utilized, injection of drugs or other chemical agents, use of ultrasonic energy to disconnect the nerves or a portion of nerves of the renal arteries.

If prescribed lifestyle changes do not address a patient's hypertension, their HCP will typically prescribe drug therapy to treat their hypertension. There are multiple classes of pharmaceutical products that can be utilized to treat hypertension. These include vasodilators to reduce the blood pressure and ease the workload of the heart, diuretics to reduce fluid overload, inhibitors and blocking agents of the body's neurohormonal responses, and other medicaments or medications. Many times, a HCP will prescribe one or more of these products to a patient to be taken in combination in order to lower their blood pressure. However, the use of pharmaceutical products is not without their risks. Many of these products carry warnings of potential side effects. Additionally, each patient may respond differently to the products, therefore multiple office visits may be required before the right dosage and type of pharmaceutical products are selected, which leads to greater health care costs. Further still there are a number of patients who either do not respond to medication, refuse to take medication, or over time the medication no longer provides a therapeutic effect. Recently, new clinical trial data has drawn correlations between the use of diuretic pharmaceutical products to treat high blood pressure and the incident or occurrence of diabetes in those patients.

For patients who do not respond to drug therapy, there are medical devices and treatments that can be utilized to treat high blood pressure. Some of these devices involve invasive surgical procedures including the implantation of a permanent medical device within a patient's artery to impart a force at a specific location within the artery which then may cause a lowering of blood pressure. However, these devices are relatively new or are still under development and have not been proven over a long period of time. Also, since the device is a permanent implant, there is always the possibility of complications during the implantation process or infections related to the implantation.

In addition to renal denervation, another type of medical device and procedure being developed is the use of an ablation catheter to denervate the carotid body, specifically the chemoreceptors of the carotid body. Similar to the device and procedure described above, this device permanently causes a disconnection between the chemoreceptors and the nervous system/brain. The long term effects are unknown, and additionally, other nerves may be destroyed or disconnected during the procedure which may lead to other side effects.

Another type of invasive medical procedure to treat hypertension being developed is to use an ablation catheter placed within the renal artery, where a series of energy pulses are performed to ablate (sever) the nerves surrounding the artery, thereby effectively disconnecting the nerves of the kidney from the body. This procedure results in a permanent and non-reversible change to the patient's nervous system, this procedure is being referred to as renal nerve ablation or renal denervation. The long-term effects of such a permanent treatment are unknown at this time as this approach is relatively new on the market. Recently published data has shown that not all patients respond to this surgical procedure; that is, after the procedure, some of the patients show little to no changes in their blood pressure. This may be concerning as now these patients have had their renal arteries permanently disconnected from the nervous system leading to their kidneys, which may lead to long term effects which are unknown at this time. Additionally, the costs associated with an invasive medical procedure are not insignificant, only to prove that the procedure had no effect, thus, instead of potentially lowering the cost of treatment for these patients, the cost of treating their hypertension was significantly added to.

Additionally, the recently published data also shows that patients who respond to renal denervation may still remain hypertensive. Thus, the renal denervation procedure may not be a “cure,” instead it may be seen as an adjunctive therapy, as such these patients may remain on drug therapies or are recommended to remain on drug therapy after having undergone renal denervation.

Yet another invasive surgical approach to address hypertension is a combination of a device and a pharmaceutical product, wherein a catheter with a needle disposed near its distal end are placed within the renal artery. Once in position, a liquid pharmaceutical product is injected into the wall of the artery or into the area surrounding the wall of the artery, whereby the pharmaceutical product is designed to chemically ablate the renal nerves. Here again, this treatment procedure is considered to be a permanent alteration of the nerve traffic between the brain and kidney, whereby the nerves are permanently severed. Long term efficacy of the severing of the renal nerves is unknown. Additionally, long term effects of the procedure are also unknown.

Recent clinical study results have shown that in order for a denervation procedure to be effective the targeted nerves need to be actually denervated. To properly perform the surgical procedure, the doctor places the catheter in the correct location and then using the catheter make a number of lesions. Early clinical data shows that 6-9 lesions per artery appears to be effective as long as the denervations are circumferential along the axis of the artery, encompassing 360 degrees of denervation and cutting off the information flow from the target area to the brain and vice-versa. However, to perform such procedures, a doctor needs to be well trained and versed on the catheter system as well as the patient's anatomy. Before, during or after the denervation the doctor does not have any feedback or real time data to know if the procedure was properly performed.

Therefore there is a need for a device that is capable of detecting nerve activity. In accordance with the present disclosure there is provided a catheter and methods, wherein the catheter is configured of being capable of measuring nerve activity, further still, in some embodiments, the catheter is configured to elicit nerve activation and nerve response.

### SUMMARY

In accordance with the present disclosure there is provided a device, the device configured to produce, for example, acoustic or vibrational or acoustic vibrational energy, comprising an elongated shaft member, an activation member that includes a vibration source, and a measurement member. In some embodiments, a method of detecting nerve activity is disclosed. In some embodiments, the method comprises inserting a catheter into the lumen of a mammal having a sidewall. In some embodiments, the catheter comprises an activation member and a measurement member spaced apart on the catheter from the activation member, where the measurement member is configured to detect energy from a nerve of interest proximate or within the sidewall of the lumen. In some embodiments, the method further comprises delivering energy from the activation member toward the nerve of interest adjacent to the lumen where the catheter is placed, wherein the activation member produces vibratory energy to activate the nerves. In some embodiments, the method further comprises detecting nerve activity using the measurement member when the measurement member measures a signal greater than 0.

### BRIEF DESCRIPTION OF THE DRAWINGS

FIG. 1 is an exemplary embodiment of a catheter in accordance with the present disclosure;

FIG. 2 is an exemplary embodiment of an alternative embodiment of a catheter in accordance with the present disclosure;

FIGS. 3A-E show an alternative embodiment of a catheter in accordance with the present disclosure;

FIG. 4 is an exemplary embodiment of an alternative embodiment of a catheter in accordance with the present disclosure;

FIG. 5 is an exemplary embodiment of an alternative embodiment of a catheter in accordance with the present disclosure; and

FIG. 6 is an exemplary embodiment of an alternative embodiment of a catheter in accordance with the present disclosure.

FIGS. 7A-B show an alternative embodiment of an activation member in accordance with the present disclosure.

FIGS. 8A-D show an alternative embodiment of an activation member in accordance with the present disclosure.

FIGS. 9A-B show an alternative embodiment of an activation member in accordance with the present disclosure.

FIG. 10 shows an alternative embodiment of an activation member in accordance with the present disclosure.

FIG. 11 shows an alternative embodiment of an activation member in accordance with the present disclosure.

FIG. 12 shows an alternative embodiment of a motor and activation needle in accordance with the present disclosure.

FIG. 13 shows an alternative embodiment of a motor and activation member in accordance with the present disclosure.

FIGS. 14A-C show a measurement/activation member in accordance with the present disclosure.

FIG. 15 shows a measurement member in accordance with the present disclosure.

FIG. 16 shows the activation and measurement of renal nerves using the device disclosed herein.

FIG. 17 shows an alternative embodiment of the methods disclosed herein where measurement is achieved through other means.

FIG. 18 show alternative embodiments of the methods disclosed herein where vibration is applied outside the body.

### DETAILED DESCRIPTION

The following detailed description illustrates embodiments of the disclosure by way of example and not by way of limitation. The description enables one skilled in the art to make and use the disclosure and describes several embodiments, adaptations, variations, alternatives, and uses of the disclosure, including what is presently believed to be the best mode of carrying out the disclosure.

This written description uses examples to disclose the embodiments of the present invention, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

Disclosed herein, in some embodiments, are catheters and methods of use for measuring nerve activity within a mammal. Nerves are normally activated and detected via electrical activity. However, in the case of ultrasound or radio-frequency denervation, the nerves and surrounding area are constantly being bombarded by electrical energy, heat energy etc. Thus, some embodiments of the present disclosure relate to activation of nerve fibers by acoustic-vibro interrogation, vibrations or acoustic vibrations. The accompanying stimulus can be detected electrically without affecting or effecting the denervation catheter. High frequency (ultrasound) does not travel far within an artery wall and is highly focused. Low frequency (sound or infrasound) travels long distances and is a wide-band emission source. Not to be limited by theory, use of a localized low frequency emission system (e.g., less than about 200, 190, 180, 170, 160, 150, 140, 130, 120, 110, 100, or less Hz in some embodiments may stimulate the efferent and afferent nerves within close proximity and as such can detect if the nerves are firing before denervation, and again after denervation. This type of acoustic stimulus and electrical detection could be used, for example, in arteries, veins, in lungs, in the heart, for pulmonary or other anatomic systems for denervation. The nerves could be, for example, autonomic nerves such as sympathetic or parasympathetic nerves. In some embodiments, the nerves may be sympathetic renal nerves.

The embodiments disclosed herein may utilize the same or similar construction for the catheter as shown and described herein. In some embodiments, catheters may be embodied as an “over-the-wire” catheter; that is, the catheter shaft includes at least one lumen extending along the length of the shaft of the catheter, wherein the lumen is configured to receive a guidewire therethrough. Alternatively, the catheter may be embodied as a “rapid-exchange” design. Rapid-exchange catheters include a lumen in the distal portion of the catheter shaft which is configured to receive a guidewire. Unlike over-the-wire catheters, rapid exchange catheters do not have a guidewire lumen extending along the entire length of the catheter shaft. Further still, the catheter may be designed such that a guidewire lumen is not necessary, instead the catheter may be guided to the location for use utilizing other known devices and methods such as through the use of a guiding sheath.

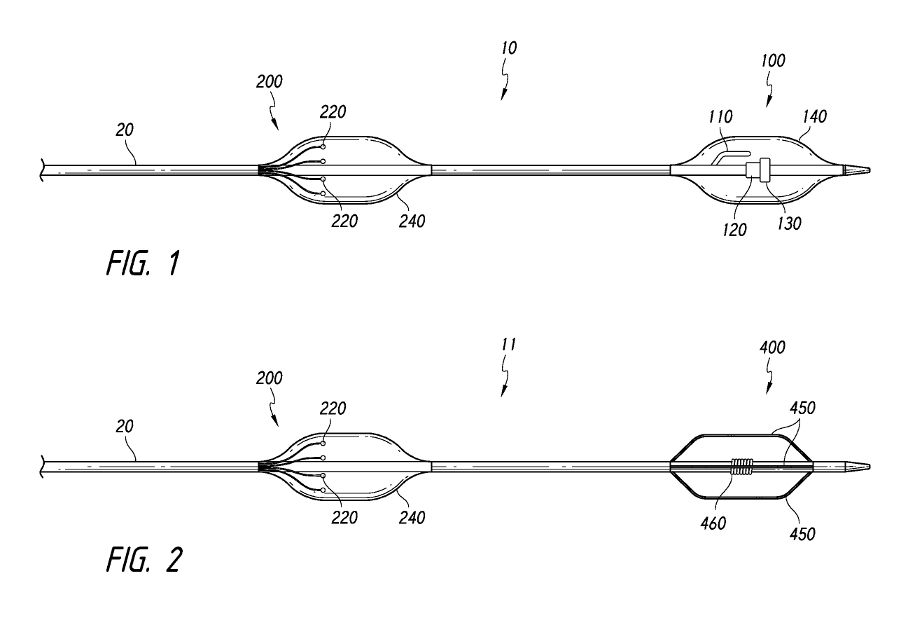

Referring now to FIG. 1, there is shown an exemplary embodiment of a catheter 10 in accordance with the present disclosure. The catheter 10 includes an elongated body 20, a measurement member 200 and an activation member 100, the activation member 100 being disposed distal the measurement member 200.

It should be appreciated that in each of the disclosed embodiments, the activation member(s) may alternatively be disposed either proximal and/or distal to the measurement member, or spaced apart axially and/or radially from the measurement member. In addition, the space between the activation member and measurement member may be varied as desired or necessary.

As shown in FIG. 1, the measurement member includes an expandable member 240. The expandable member 240 may be embodied as an inflatable balloon, wherein the catheter shaft 20 includes an inflation lumen in fluid communication with the balloon. Alternatively, the expandable member 240 may be constructed of metal, plastic, or another biocompatible material, or any combination thereof. The expandable member 240 may be a self-expanding design, where a protective sheath (not shown) or a retention member (not shown) is retracted or pushed forward allowing the self-expanding expandable member 240 to expand. In another embodiment, the expandable member 240 may be expanded using mechanical means (not shown) for example, the proximal end of the expandable member 240 can be affixed to the catheter shaft 20 and the proximal end be configured to slide along the shaft 20, a pull wire or similar structure would be attached to the distal end of the expandable member, wherein when a pull force is applied to the pull wire the expandable member 240 would be expanded. It shall be understood that the arrangement of the pull wire and the fixed end of the expandable member 240 can be reversed. In addition, in alternative embodiments a push wire may be used. In yet alternative embodiments, the expandable member may be expanded through the rotation of the wire. The measurement member 200 further includes at least one measurement sensor 220 associated with the expandable member 240. The measurement sensor 220 may be embodied as an electrode configured to measure electrical activity. The electrode/measurement sensor 220 may be embodied as a flexible circuit board, or as a discrete sensor disposed on the expandable member 240. The measurement sensor 220 is electrically coupled either with wires or wirelessly to another location, such as the proximal end of the catheter 10, wherein a separate measurement device (not shown) can be electrically coupled to the measurement sensor(s) 220. The measurement device may be a data recording device, a computing device or similar.

In some embodiments, the catheter 10 further includes an activation member 100 disposed adjacent to the distal end of the catheter shaft 20. As shown in FIG. 1, the activation member includes an expandable member 140. The expandable member 140 can be constructed in a similar manner as that described above with regard to the expandable member 240. In the embodiment shown, the expandable member 140 is embodied as an inflatable balloon. The balloon may be inflated with a fluid such a saline, and/or may be inflated with a gas such as CO2. The activation member 100 can further include a tuned arm 110 and a rotatable member, which can be an eccentric member 130 in some embodiments. The eccentric member 130 is coupled to a rotation source 120. The rotation source may be, for example, an electric motor disposed within the area of the balloon, or it may be embodied as a driveshaft which runs the length of the catheter shaft wherein the driveshaft is coupled to a rotation source. It is contemplated that other rotational sources may be utilized, for example the rotation source 120 may be embodied as a turbine, wherein fluid is utilized to inflate the balloon activates the rotation source. Further still, if the expandable member is embodied as a balloon, the inflation medium (saline or contrast) can be utilized as a lubricant for the spinning driveshaft.

In some embodiments, the activation member could utilize one, two, or more of the following energy modalities: RF energy (e.g., using a monopolar or bipolar electrode), coherent light energy or incoherent light energy (e.g., using an optical light source), thermal energy (e.g., heat or cold), microwave energy (e.g., using a microwave antenna), ultrasound energy, plasma energy (via ignition of gas that is conducted either directly outside of the balloon or through the transfer heat to the surface of the balloon), sound energy, mechanical energy (e.g., a fluid jet), magnetic energy, electrical energy, and others.

In use, the catheter 10 is advanced into the renal artery or other artery in which it is desired to measure nerve activity. The expandable member 140 of the activation member is expanded and the expandable member 240 of the measurement member 200 is expanded, either at the same time or sequentially. The rotation source 120 is activated, wherein the eccentric member 130 rotates about the shaft 20 of the catheter 10. The eccentric member 130 is configured to strike the tuned arm 110; the tuned arm 110 emits a vibration in response to the strike by the eccentric member 130. The speed of the rotation source 120 can be adjusted to increase or decrease the frequency at which the eccentric member 130 strikes the tuned arm 110. Simultaneously, the measurement sensor(s) 220 which are in electrical contact with the wall of the lumen in which the catheter is disposed within receive electrical signals generated by the nerves firing in response to the vibrations generated by the tuned arm 110.

The vibrations of the tuned arm 110 cause a response from the nerves, this nerve response can be then detected by the measurement sensor(s) disposed on the measurement member 200. By activating and then detecting nerve activity, an HCP, who for example just performed a renal denervation procedure, can then determine if the procedure has been properly performed or if additional energy needs to be delivered for further nerve denervation.

It shall be understood that although the disclosure herein in some embodiments relates to a separate stand alone catheter, it is contemplated that the structures and concepts as disclosed herein can be incorporated into a renal denervation catheter or any other catheter, such as a balloon catheter, stent delivery catheter, etc. For example, a combination ablation-nerve activity detection catheter could include, for example, an ablation member incorporating one or more ablation elements configured to ablate one or more nerves, a measurement member spaced apart from the ablation member, and an activation member spaced apart from the measurement member, which is configured for vibration in some embodiments. The ablation element could include, for example, RF, microwave, ultrasound, cryoablation, thermal ablation, chemical ablation, mechanical ablation (e.g., via cutting) or other modalities. In some embodiments, the ablation member is a different modality, such as a different energy modality, or energy characteristic (such as a different frequency) than the ablation member, such as a non-vibrational energy member. For example, in some embodiments the ablation member can be configured to transmit RF energy while the activation member can be configured to transmit vibrational energy.

Referring now to FIG. 2 there is shown an alternative embodiment of a catheter in accordance with the present disclosure. As shown in FIG. 2, the catheter 11 includes an elongated catheter shaft 20, a measurement member 200 and an activation member 400. In the embodiment shown in FIG. 2, the measurement member 200 can be embodied as that described above with regard to FIG. 1. The activation member 400 includes an expandable member 450, wherein the expandable member 450 is embodied as a cage design with a plurality of interconnected struts separated by open cells. The cage may be configured as a unitary member cut from a single tubular member or a sheet member, or the cage may be embodied as a plurality of separate members. The catheter 11 further includes a flex member 460 disposed within the shaft 20 of the catheter 11. In use, the flex member 460 is coupled to a pull wire (not shown), a force is applied to the pull wire thereby compressing the flex member 460 whereby the expandable member 450 expands. The force on the pull wire can be embodied as a cyclic force which causes the expandable member to expand and contract quickly, thereby causing an intermittent force to be applied to the wall of the vessel/artery/lumen. Additionally, the expansion can be further controlled by adjusting or changing the distance that the pull wire is pulled. As described above, the expandable member 240 of the measurement member 200 is expanded, wherein the measurement sensor 220 is in contact with the wall of the vessel/artery/lumen. In some embodiments, the measurement sensor can advantageously rest against the intimal (intraluminal) surface of the vessel wall and need not penetrate through said wall. As described above with regard to FIG. 1, in use, the catheter of FIG. 2 functions in a similar manner, wherein the measurement sensor(s) 220 measure electrical activity of the nerves in response to the expansion/contraction of the expandable member 450.

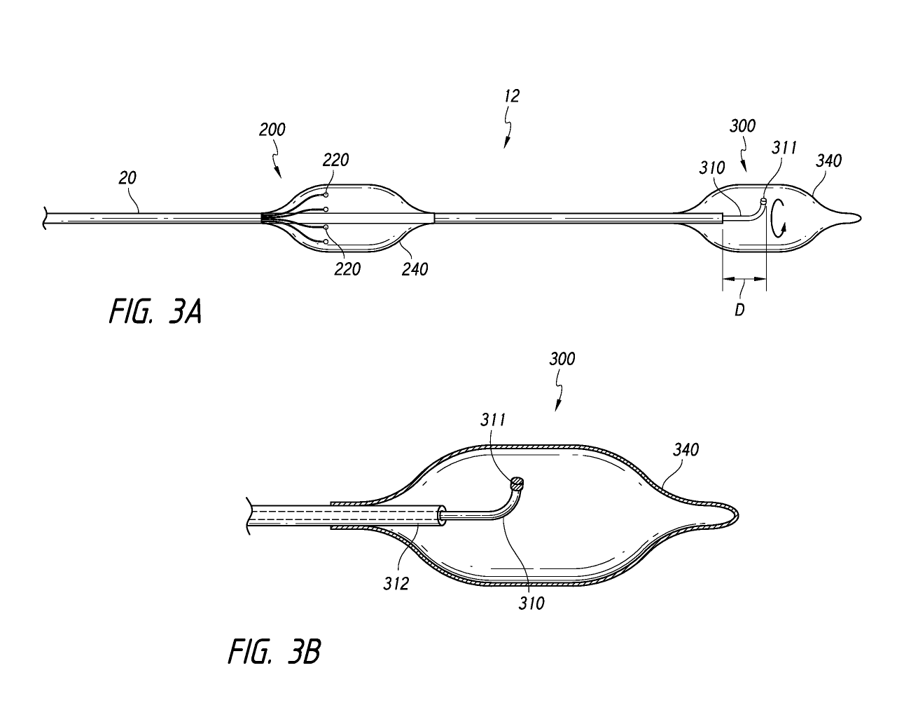

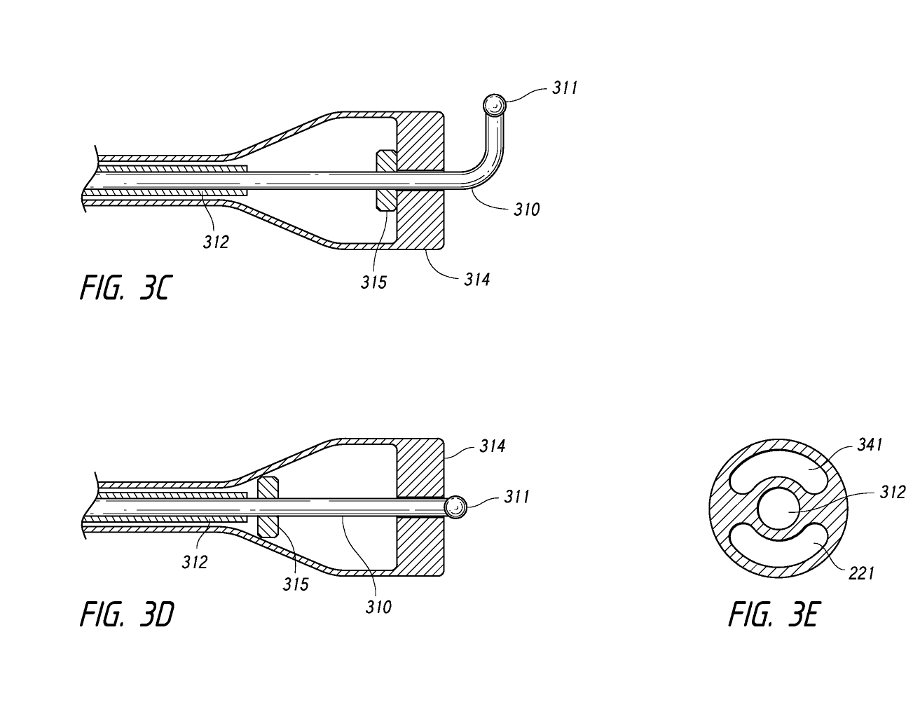

Referring now to FIGS. 3A-E, there is shown yet another alternative embodiment of a catheter in accordance with the present disclosure. As shown in FIG. 3A, the catheter 12 utilizes a measurement sensor 200 as described herein with regard to the other embodiments of the present disclosure. As shown in FIGS. 3A and 3B, the catheter 12 further includes an activation member 300, and the activation member includes an expandable member 340. The expandable member 340 can be embodied as a balloon member or other structure as described above. In addition to the expandable member 340, the activation member further includes a rotation member 310, the rotation member being disposed within a lumen of the catheter 12 and configured to rotate within the expandable member 340. The rotation member 310 can be coupled to a motor (not shown) disposed within a lumen of the catheter shaft. Alternatively, the rotation member 310 may be coupled to a driveshaft 312 disposed within a lumen of the catheter. The driveshaft 312 can then be coupled to a rotational member, such as a motor disposed at the proximal end of the catheter or separate from the catheter. The rotational speed of the rotation member 310 can be adjusted to increase or decrease the amplitude of vibration produced by the rotation member. Additionally, a weight 311, such as a metal ball, can be added to the end of the rotation member 310, wherein the weight can be adjusted to tune the vibrations (e.g., increase or decrease the frequency) produced by the rotation member 310. In some embodiments, the fluid inside the expandable member 340 can travel back inside the catheter 12, thus lubricating the driveshaft 312. In some embodiments, the end of the catheter 12 may include a bushing or bearing surface for the rotation member 310 to rotate against. In some embodiments, the driveshaft 312 and/or rotation member 310 may be shaped as a helix or coil. Additionally, in some embodiments, the driveshaft 312 and/or rotation member 310 may be a low friction material such as nylon or polyimide, and/or may be braided. In some embodiments, the driveshaft 312 and/or rotation member 310 may be comprised of a trilayer of material with, for example, a nylon outer layer, a middle layer, and an inner PTFE layer.

FIG. 3C shows the rotation member 310 extended axially distally into the expandable member 340 and FIG. 3D shows the rotation member 310 pulled back proximally within the lumen of the catheter 12. The rotation member 310 can be advanced and retracted by changing the distance D between the end of the catheter shaft and the rotation member 310. By doing so, the amplitude of the vibrations produced by the rotation member 310 can be adjusted. For example, in some embodiments, the amplitude is increased as the rotation member 310 is extended into the expandable member 340. In some embodiments, the distance of the rotation member 310 within the expandable member 340 changes, but the rotation member 310 does not rotate. In some embodiments, a stopper 315 may be crimped, glued, welded, or otherwise attached to the driveshaft 312 so that the rotation member 310 may only be extended into the expandable member 340 until the stopper 315 reaches the end of catheter 12. Similarly, when the rotation member 310 is pulled back into the catheter 12, the weight 311 and/or stopper 315 will prevent the rotation member 310 from being pulled too far back into the catheter 12. In some embodiments, a bushing 314, such as a polyoxymethylene (POM) bushing inside the expandable member 340 can control how far the rotation member 310 may be extended or pulled back. FIG. 3E shows a cross-section of an embodiment of the catheter 12 with separate driveshaft 312, inflation lumen 341, and electrode lumen 221. The inflation lumen 341 can carry the inflation media through the catheter 12 until it reaches the expandable member 340. Meanwhile, the electrode lumen 221 can connect to the electrodes 220 in the measurement member 200. In alternative embodiments, the electrode lumen 221 may also connect to electrodes near the proximal end of the expandable member 340 (not shown).

The catheter 12 can be utilized in a similar manner as the previous catheter designs as previously described herein. The rotational member is utilized to produce vibrations and the measurement sensor(s) 220 are utilized to measure electrical nerve activity.

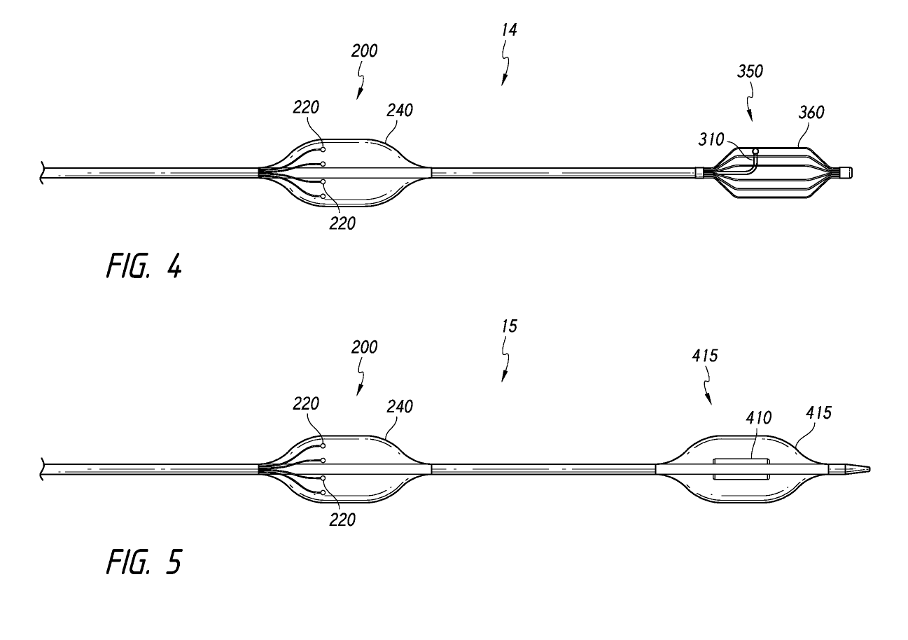

Referring now to FIG. 4, there is shown yet another alternative catheter design in accordance with the present disclosure. The catheter 14 of FIG. 4 utilizes a measurement member 200 as shown and described herein with regard to the other embodiments. The catheter 14 includes an activation member 350, wherein the activation member 350 includes a cage member 360 and a rotational member 310 similar to that described above with regard to FIG. 3. The cage member 360 can be constructed, for example, of a material such as music wire, or cut from a tubular member to form a cage member. In the embodiment depicted in FIG. 3, the cage member 360 is configured to be embodied as a tuned member; that is, the cage member 360 is intended to function as a “music box”. The rotation member 310, when rotated, strikes each of the struts of the cage member 360, wherein when struck, the struts produce vibrations. These vibrations can be directly transmitted to the lumen or the vibrations may be transmitted through fluid within the lumen.

Referring now to FIG. 5 there is shown another alternative embodiment of a catheter in accordance with the present disclosure. As shown in FIG. 5, the catheter 15 includes a measurement member 200 similar to that described herein with regard to the other embodiments. The catheter 15 further includes an activation member 415. The activation member includes an expandable member 440, wherein the expandable member 440 may be a balloon or an expandable cage as described previously herein. The activation member 415 further includes an activation device 410. The activation device 410 may be a piezo speaker, a haptic speaker, an ultrasonic transducer or an electroactive polymer. Alternatively, the activation device 410 may contain a taut string, similar to a guitar string, that is configured to vibrate or otherwise creating acoustic energy when activated. In some embodiments, the activation device 410 may include more than one piezo speaker, haptic speaker, ultrasonic transducer, electroactive polymer, string, or other member/speaker. For instance, in some embodiments the activation device 410 may include two or more piezo speakers. Such piezo speakers may, for example, create a low frequency binaural beat in order to deliver energy to the surrounding lumen/nerve. For example, the binaural beat can be created from a first frequency tone and a second frequency tone, wherein the difference between the first frequency and the second frequency is about or less than about 30 Hz, 25 Hz, 20 Hz, 15 Hz, or less. The activation device 410 may be electrically coupled to an energy source such as a frequency generator, a power supply, a music source or similar devices capable of activating and causing the activation device 410 to produce vibration or sounds.

The catheter 15 is utilized in a manner similar to that previously described herein, wherein the activation member is configured to apply vibrational energy within a lumen and the measurement member is configured to detect and capture electrical nerve activity in response to the vibrational energy.

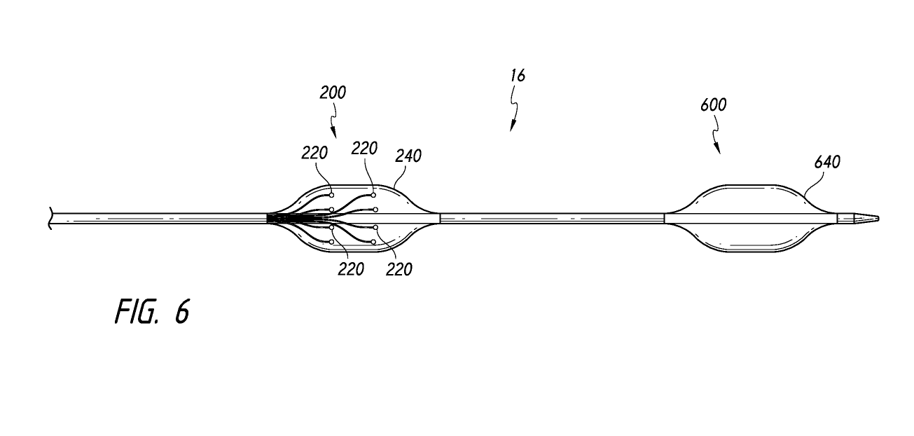

Referring now to FIG. 6, there is shown yet another alternative catheter in accordance with the present disclosure. As shown in FIG. 6, the catheter 16 includes a measurement member 200 as described herein. Additionally, catheter 16 includes an activation member 600, wherein the activation member 600 is embodied as a balloon 640. The balloon 640 is in fluid communication with an inflation lumen (not shown) disposed within the shaft of the catheter 16, wherein the other end of the inflation lumen is coupled to an inflation/deflation source such as a syringe or pump. The inflation/deflation source is utilized to cause the balloon to inflate and deflate, by controlling the time interval of the inflation/deflation cycle a vibration can be generated by the balloon, wherein the vibration is transmitted to the lumen that the balloon is disposed within. As described herein the vibration caused by the balloon activates the nerves wherein the nerve activity can then be detected utilizing the measurement member.

The balloon 640 or any of the other catheter embodiments that utilize a balloon for the expandable member may also be inflated with a gas such as CO2 or a liquid as described herein. By rapidly controlling the inflation medium the balloon can be made to oscillate, thereby producing an acoustic vibration which can be utilized to activate the nerves. After fully inflating the balloon, the acoustic vibrations are produced by rapid partial deflation of the balloon and rapid partial inflation of the balloon. By controlling flow rate the amplitude of the acoustic vibrations can be controlled and by controlling the switching time between partial inflation and partial deflation the frequency of the acoustic vibrations or vibrations can be controlled.

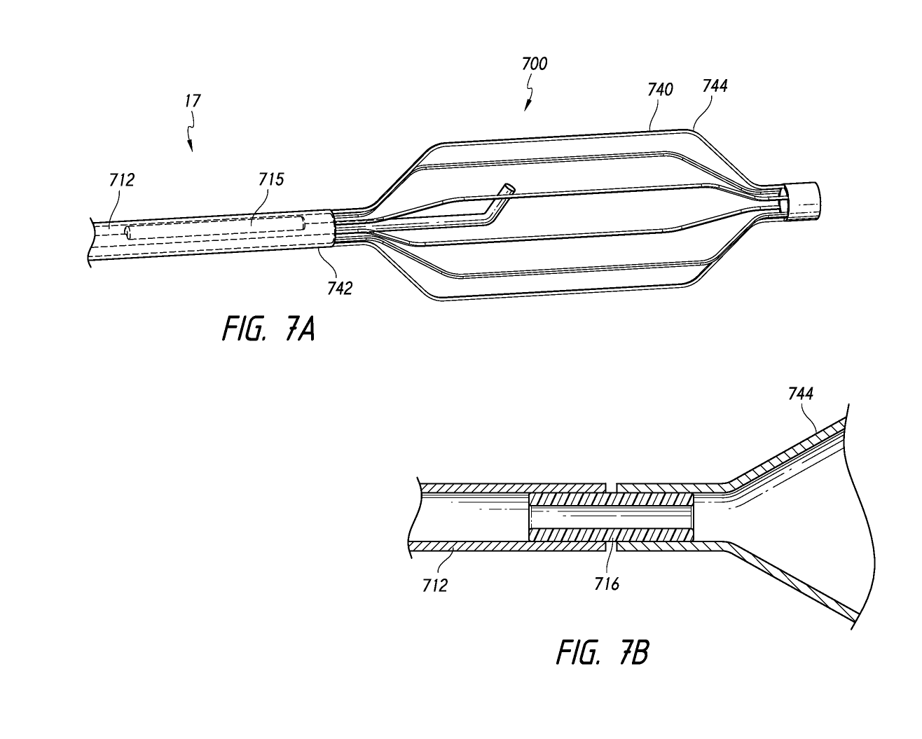

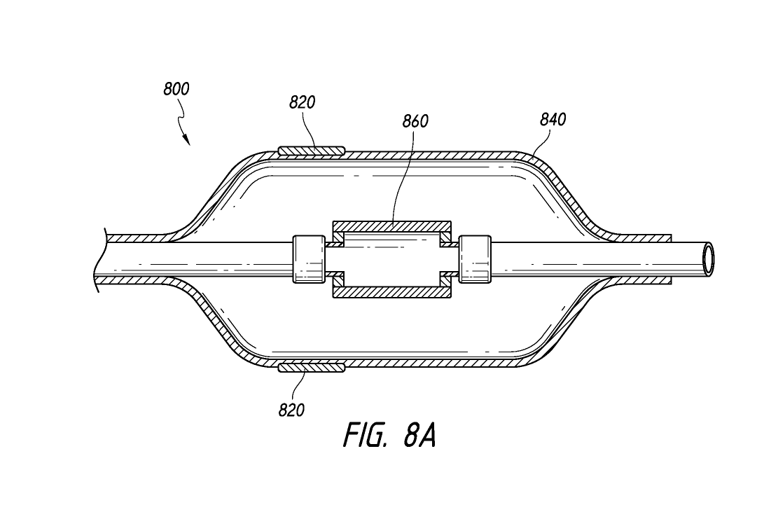

Referring now to FIG. 7A, there is shown an alternative activation member in accordance with the present disclosure. As shown in FIG. 7A, the activation member 700 may include a balloon 740 comprised of an outer shaft 742 that is able to move to collapse/expand a collapsible cage 744. In some embodiments, the collapsible cage 744 may be comprised of nitinol. A driveshaft inside of an inner shaft 712 may transport fluid or gas from the shaft of the catheter 17 to the balloon 740. This transportation of fluid or gas may further drive the balloon 740 to expand. In some embodiments, the inner and outer shafts may be comprised of nylon material. Alternatively, the inner shaft 712 may be comprised of a low friction material, and/or the material may be braided. The inner shaft 712 may be made of a single material such as nylon or polymide or a trilayer material with, for example, the outer layer being nylon, a middle layer, and an inner layer of PTFE. The driveshaft may be coiled or helical in order to transport fluid. In some embodiments, the driveshaft may further comprise a small eccentric weight 715, and the eccentric weight may aid in the expansion/collapse of the balloon 740 and collapsible cage 744 as it extends from/pulls back into the outer shaft 742. As shown in FIG. 7B, in some embodiments, the inner shaft 712 may further include a bushing 716 made out of POM or some other suitable material to allow the driveshaft to slide into the collapsible cage 744. The distance D that the driveshaft is displaced into the collapsible cage 744 may control the amplitude of vibratory energy delivered to the balloon 740. In some embodiments, electrodes on the proximal end of the activation member 700 (not shown) may additionally be employed to measure nerve activity.

It is contemplated that the alternative activation member embodiments described herein may be used alone or in combination with a separate, spaced apart measurement member on the catheter.

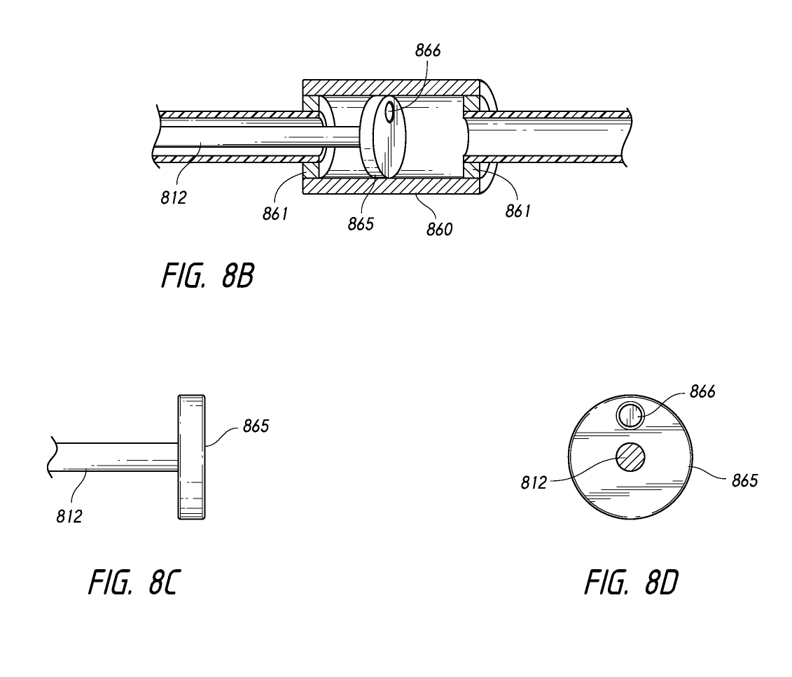

Referring now to FIGS. 8A-D there is shown an alternative activation member 800 embodied as an expandable balloon 840 with an internal cage 860. The internal cage 860 may be made out of POM or some other suitable material. The expandable balloon 840 may also include proximal electrodes 820 for the concurrent measurement of energy.

As shown in FIG. 8B, an eccentric disk 865 is positioned inside of the internal cage 860 and may be moved proximally or distally by the driveshaft 812. The driveshaft 812 may be comprised of a low friction material and/or the material may be braided. The driveshaft 812 may be made of a single material such as nylon or polymide or a trilayer material with, for example, the outer layer being nylon, a middle layer, and an inner layer of PTFE. Marker bands 861 may delineate the ends of the cage 860 where the driveshaft 812 enters and leaves the cage 860. A weight 866 on the eccentric disk 865 creates vibratory energy that is transmitted outside of the cage 860 and into the expandable balloon 840 and lumen walls.

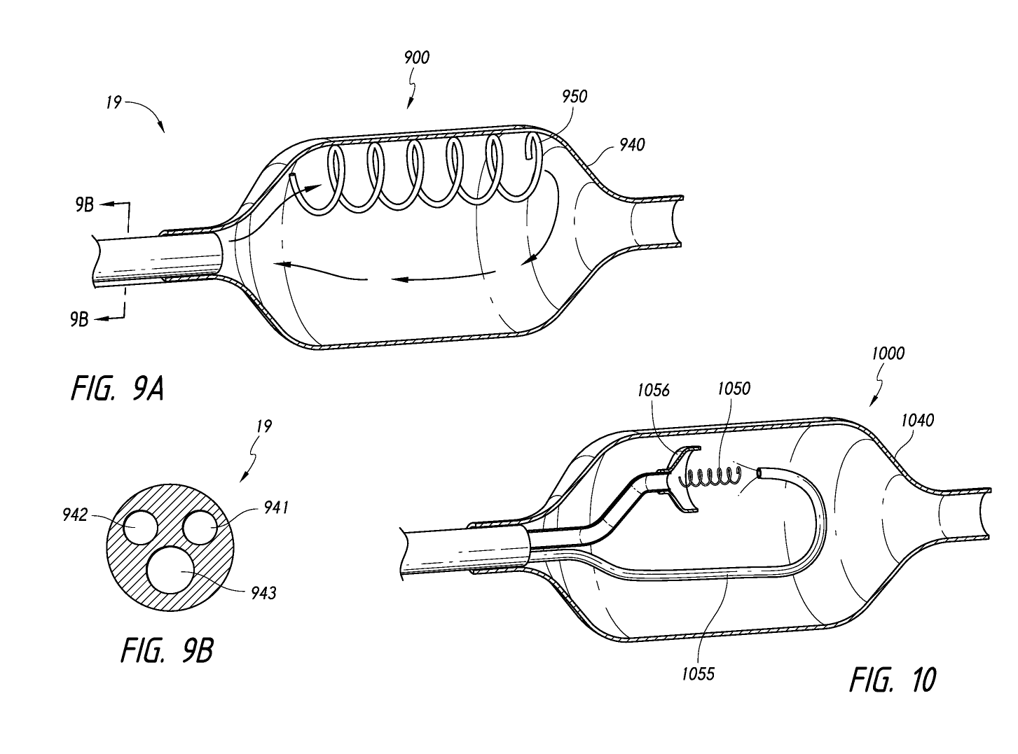

Referring now to FIGS. 9A-B, there is shown yet another embodiment of an alternative activation member 900. In this case, the activation member 900 includes a spring 950, coil, or other structure inside a balloon 940, which can be helical or another shape. Fluid may enter the balloon 940 and travel through the spring 950. This may cause turbulence in the balloon 940, creating vibratory energy in the balloon 940. By increasing or decreasing the amount of fluid that passes through the spring 950, the amount of vibratory energy created may be adjusted. FIG. 9B shows an example cross-section of the catheter 19 of such an embodiment, wherein the catheter comprises a multi-lumen shaft. In some embodiments, fluid is delivered to the balloon 940 through one lumen 941 and travels out of the balloon 940 through a second lumen 942. In some embodiments, a third lumen 943 connects to electrodes at a measurement member and/or electrode situated at the proximal end of the activation member (not shown). In some embodiments, a fourth lumen (not shown) may additionally pump fluid or gas into the balloon 940 to increase the turbulent flow of fluid and gas within the balloon 940.

Referring now to FIG. 10, there is shown yet another embodiment of an alternative activation member 1000. Similar to the embodiment shown in FIGS. 9A-B, activation member 1000 includes a balloon 1040 with a spring 1050, coil, or other structure to create turbulent flow. Fluid travels into the balloon 1040 through needle 1055 and then enters spring 1050, creating vibratory energy in the balloon 1040. In some embodiments, the fluid exits the balloon 1040 through a funnel 1056 or other vacuum suction mechanism. In alternative embodiments, the fluid exits directly back into the catheter shaft with no funnel in place.

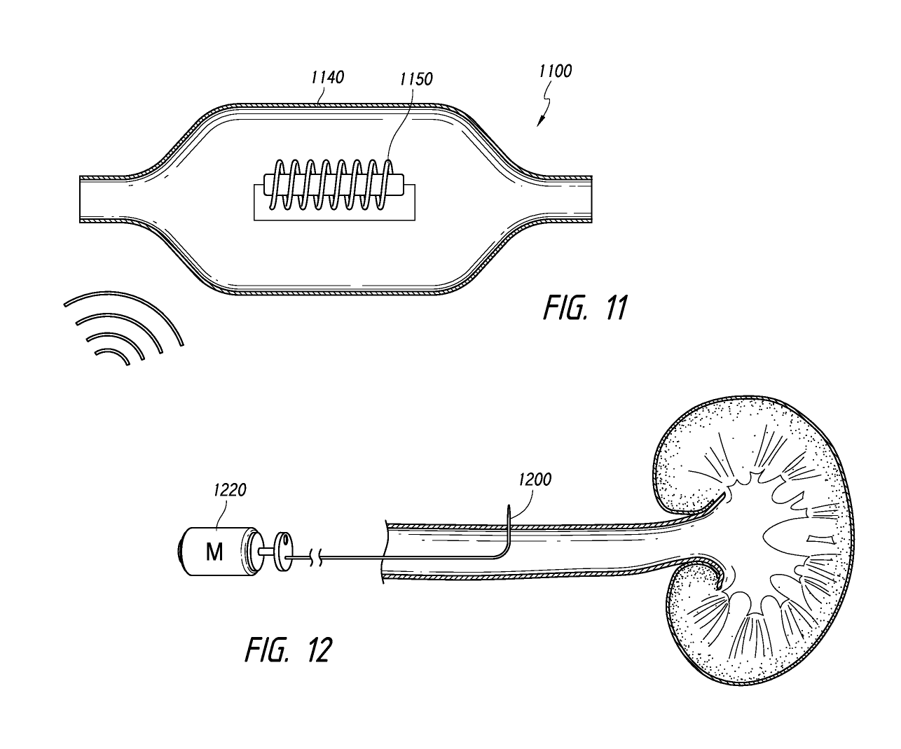

Referring now to FIG. 11, there is shown yet another embodiment of an alternative activation member 1100. In this embodiment, there is an electromagnetic coil 1150 inside of balloon 1140. The electromagnetic coil 1150 may be activated by means internal to or external to the body. For instance, heat or a magnetic field may be applied to the skin of a patient to activate the electromagnetic coil 1150, producing vibratory energy in the balloon 1140, which can be at a predetermined frequency.

Referring now to FIG. 12, there is shown an alternative embodiment in accordance with the present disclosure. In this embodiment, energy is delivered from a source external to the body, such as a motor, e.g., an eccentric motor 1220. The motor 1220 creates vibratory energy in order to vibrate a needle 1200 disposed within the lumen of a mammal. The needle 1200 may be curved to puncture into the lumen wall and thus directly contact nerves to apply vibratory energy. In some embodiments, the needle 1200 may be disposed within a catheter and extend out of the catheter to puncture the lumen, or vessel wall. In alternative embodiments, the needle 1200 is independent of a catheter, as shown.

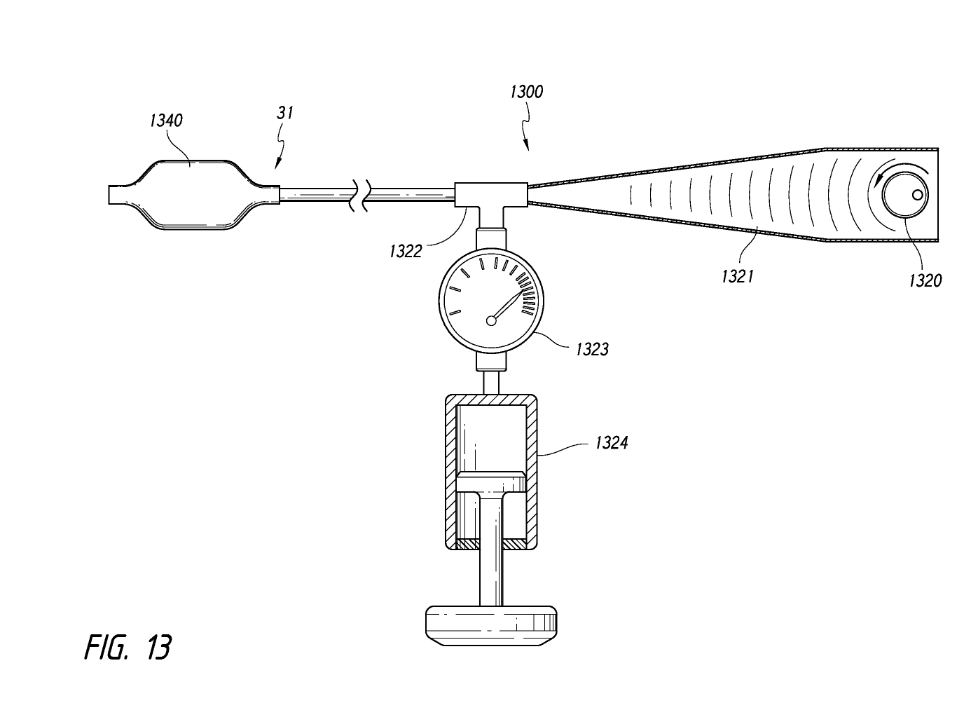

Referring now to FIG. 13, there is shown an alternative embodiment in accordance with the present disclosure. Similar to the embodiment shown in FIG. 12, this embodiment uses an external motor to control the energy delivered by the catheter 31 to a balloon 1340 situated in an artery wall or other lumen wall of a mammal. In this embodiment, a hydraulic actuation system 1300 is used. The system 1300 includes a motor 1320, such as an eccentric motor that delivers gas through a diaphragm 1321 to the actuator 1322. A pressure control valve 1323 modulates the amount of pressure received from a hydraulic pump indeflator 1324 attached to the actuator 1322. The hydraulic actuation system 1300 then delivers the appropriate amount of pressure/energy, for example in the form of shock waves through a catheter 31 to the balloon 1340.

A skilled artisan will recognize that these motor embodiments or other external energy systems may be used in conjunction with other catheter embodiments as disclosed herein.

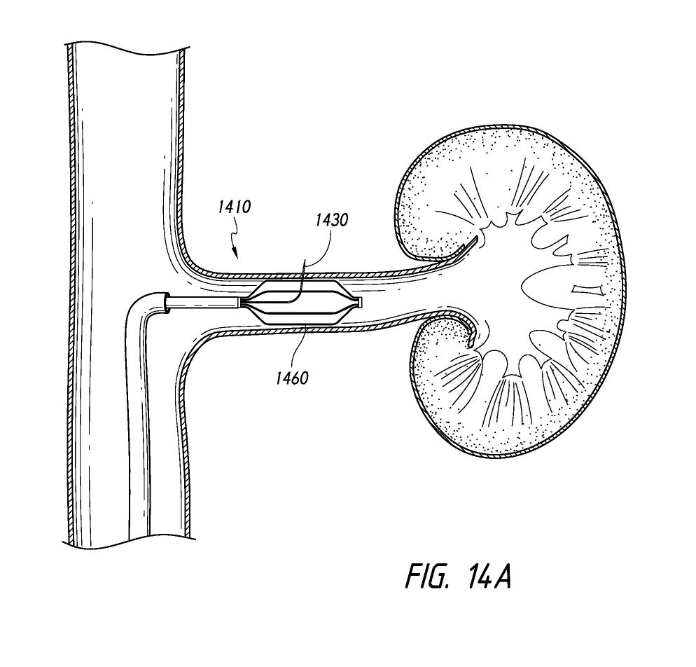

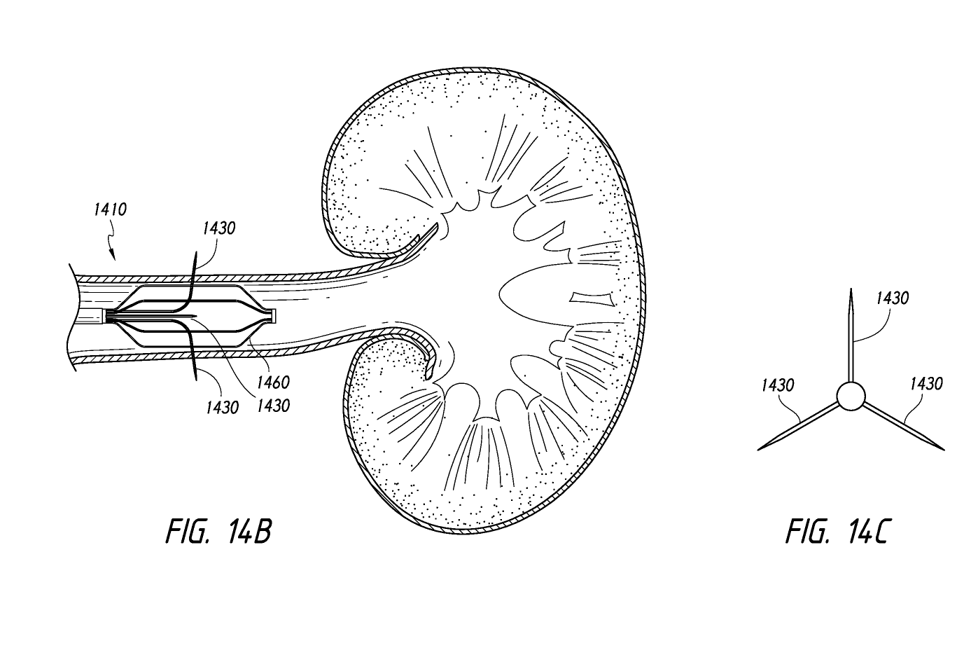

Referring now to FIG. 14A, there is shown an alternative embodiment of a member 1410 that may function as either a measurement or activation member in accordance with the present disclosure. In this embodiment, member 1410 may comprise a cage or balloon 1460. In some embodiments, the cage may be an expandable nitinol cage. In use, in contrast to non-penetrating measuring mechanisms as described elsewhere herein, a needle 1430 may be pushed from inside of the cage or balloon 1460 and puncture the lumen, or vessel, wall of a mammal so that the tip of the needle is outside of the lumen wall. As part of a measurement member, the needle 1430 may include a sensor that can measure electrical or other type of energy that has been delivered to the nerves outside of the lumen wall. In alternative embodiments, the needle 1430 may inject a dye or stain outside of the vessel wall so that nerves can be visualized. For example, SB100 dye may be injected outside of the vessel wall and viewed through other means. Alternatively, another mechanism for the detection of biomarkers may be injected, such as, for example, a labelled neurotransmitter, an antibody or protein solution and viewed. In yet other alternative embodiments, the needle 1430 may allow disrupted nerves to be visualized through an imaging modality including intravascular ultrasound or Optical Coherence Tomography (OCT). As part of a measurement member, the needle may be able to work as an energy source that may apply, for example, vibration, an electrical signal, sound, heat, cold, or drugs to nerves outside of the lumen walls. As shown in FIGS. 14B-C, alternative embodiments may include multiple needles within a cage or balloon 1460 to puncture the vessel walls. For instance, as shown in FIG. 14B-C, a cage 1460 may include three needles equally spaced around the axis of the cage 1460.

It is contemplated in accordance with the present disclosure that the activation member of some embodiments could also incorporate one, two, or more biologically active compounds, which would be injected or otherwise delivered to the lumen or nerves of a user. The active compound could be incorporated into the activation member through a lumen of the catheter, or already be previously present within the activation member during catheter insertion. For instance, the active compound could be stored in an embodiment of the activation member that includes a needle to puncture the surface of the lumen wall of a mammal. The addition of a biologically active compound can be advantageous, in some embodiments, to allow the device to create a synergistic effect via a mechanism of action (e.g., vibratory activation of nerves, for example) that could potentially be different/unrelated to increasing injection/absorption of the biologically active compound. For example, the catheter could emit energy, such as vibration/ultrasound energy to the lumen walls while the active compound may be directly injected into the nerves via a different mechanism unrelated to the application of vibratory energy.

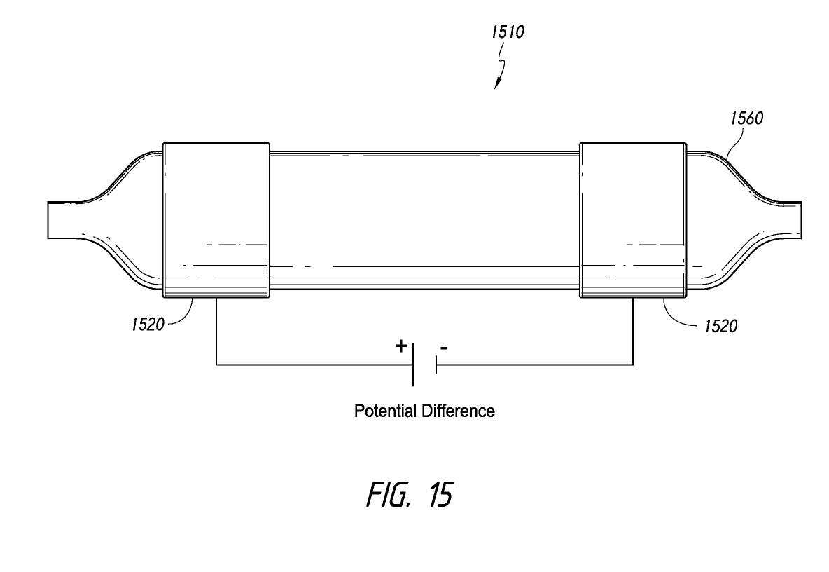

Referring now to FIG. 15, there is shown an alternative embodiment of a measurement member 1510. In this embodiment, the measurement member 1510 includes a balloon 1560 that employs bipolar or monopolar RF energy to detect a nerve signal. Electrode bands 1520 on the balloon 1560 create a potential energy difference that is used in detecting signals transmitted from the nerve through the vessel wall.

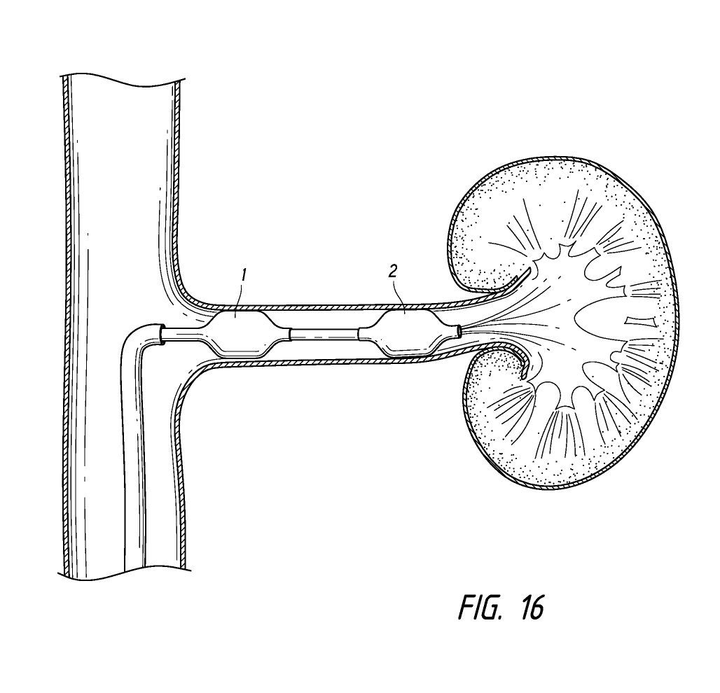

FIG. 16 shows one embodiment of the catheter system described herein within the renal artery of a mammal. In use, a catheter comprising a measurement member 1 and an activation member 2 is inserted into the renal artery. The activation member 2 delivers a form of energy to the walls of the artery in accordance with the embodiments disclosed herein. The measurement member 1 then detects the amount of energy received by the nerves external to the artery walls in accordance with the embodiments disclosed herein. The measurement member 1 sends the received signal through the catheter to be analyzed by a processor, which may determine if the nerve is active or, for example, has been ablated.

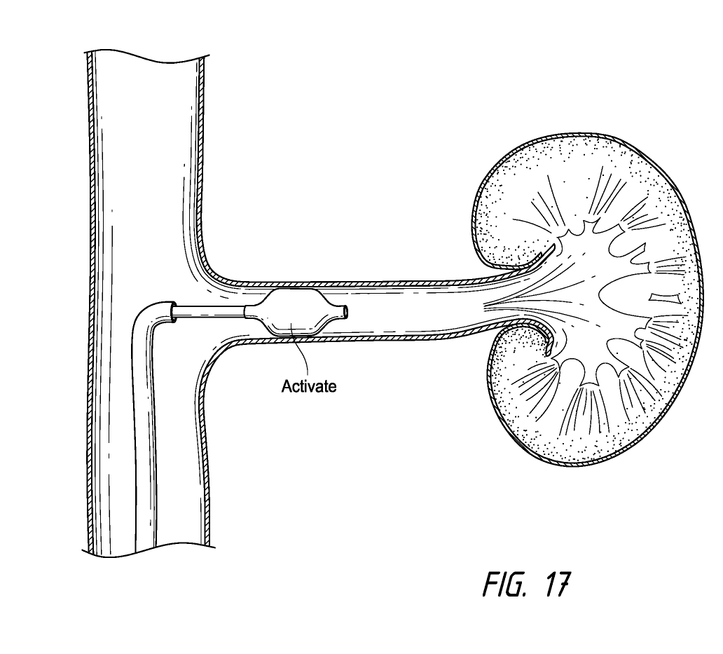

FIG. 17 shows an alternative embodiment of the catheter system described herein. In this embodiment, an activation member in accordance with any of the embodiments disclosed herein delivers energy to the nerves external to the renal artery. For example an activation member may deliver vibration/sound, temperature, light, drugs, pressure, or some other form of energy to the nearby nerves. Alternative forms of measurement are then used to determine whether the nerve remains active or has been ablated. For example, in some embodiments, after activation, vasoconstriction is measured using contrast medium, IVUS, a flow wire, an optical fiber, or some other means to check for nerve activity.

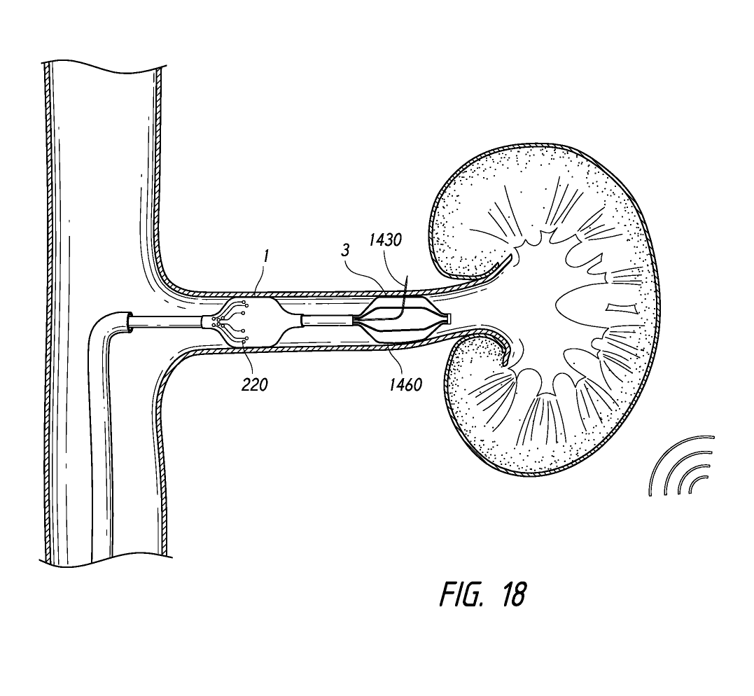

FIG. 18 show alternative embodiments of the catheter system described herein. In these embodiments, vibration or other forms of activation are applied external to the body and a measurement member 1 disposed on a catheter is inserted into the lumen to measure nerve activity. Activation may be, for example, with vibration or sound from, for example, from a vibrational pad, ultrasound probe, speaker, or some other means. In this embodiment, surface electrodes 220 of a measurement member 1 measure nerve activity in response to energy, similar to those disclosed in FIG. 1. In this embodiment, an additional or alternative measurement member is comprised of a nitinol cage 1460 with an extendable needle 1430 that measures nerve activity, similar to the cage and needle disclosed in FIG. 14. A skilled artisan will recognize, however, that any of the embodiments of one or more measurement members described herein may alternatively be used in this system embodiment.

In accordance with the present disclosure, the catheters described herein are intended to be utilized to measure nerve activity. The catheters are designed to activate nerves using vibrational or acoustic energy and/or then measure the electrical activity of the nerves which are activated by the vibration/acoustic energy. As described above, in some embodiments the catheters are designed such that they can be utilized before a denervation procedure to establish a baseline of nerve activity, during a denervation procedure to monitor the progress of the procedure and after the completion of the denervation procedure to determine/confirm whether the denervation procedure was effective. As described above, by utilizing vibrations or acoustic vibrations or acoustic energy, the nerve activation of some embodiments may advantageously not interfere with the electrical energy of the denervation catheters. However, it is further contemplated that a skilled artisan would not be foreclosed from using other energy sources to create nerve activation, such as pressure caused by expanding a balloon into a lumen wall, either increased or decreased temperature, magnets either alone or in combination with a biomarker or Ferromagnetic particles, nanoparticles, UV light, soundwaves at the infra, ultra, or audible level, other forms of non-vibratory energy, or other forms of acoustic or electro-acoustic vibration, etc. that are able to be measured.

In accordance with embodiments of the present disclosure, vibratory frequencies contemplated for use can range between 0 Hz to 20,000 Hz, 0 Hz and 10,000 Hz, 0 Hz and 5,000 Hz, 0 Hz and 2,500 Hz, 0 Hz and 1,750 Hz, 0 Hz and 875 Hz, 0 Hz and 435 Hz, 0 Hz and 200 Hz, 0 Hz and 150 Hz, 1 Hz and 150 Hz, 2 Hz and 150 Hz, 3 Hz and 150 Hz, 4 Hz and 150 Hz, 5 Hz and 150 Hz, 6 Hz and 150 Hz, 7 Hz and 150 Hz, 8 Hz and 150 Hz, 9 Hz and 150 Hz, 10 Hz and 150 Hz, 11 Hz and 150 Hz, 12 Hz and 150 Hz, 13 Hz and 150 Hz, 14 Hz and 150 Hz, 15 Hz and 150 Hz, 16 Hz and 150 Hz, 17 Hz and 150 Hz, 18 Hz and 150 Hz, 19 Hz and 150 Hz, 20 Hz and 150 Hz, 21 Hz and 150 Hz, 22 Hz and 150 Hz, 23 Hz and 150 Hz, 24 Hz and 150 Hz, 25 Hz and 150 Hz, 26 Hz and 150 Hz, 27 Hz and 150 Hz, 28 Hz and 150 Hz, 28 Hz and 150 Hz, 29 Hz and 150 Hz, 30 Hz and 150 Hz, 31 Hz and 150 Hz, 32 Hz and 150 Hz, 33 Hz and 150 Hz, 34 Hz and 150 Hz, 35 Hz and 150 Hz, 36 Hz and 150 Hz, 37 Hz and 150 Hz, 38 Hz and 150 Hz, 39 Hz and 150 Hz, 40 Hz and 150 Hz, 41 Hz and 150 Hz, 42 Hz and 150 Hz, 43 Hz and 150 Hz, 44 Hz and 150 Hz, 45 Hz and 150 Hz, 46 Hz and 150 Hz, 47 Hz and 150 Hz, 48 Hz and 150 Hz, 49 Hz and 150 Hz, 50 Hz and 150 Hz, 51 Hz and 150 Hz, 52 Hz and 150 Hz, 53 Hz and 150 Hz, 54 Hz and 150 Hz, 55 Hz and 150 Hz, 56 Hz and 150 Hz, 57 Hz and 150 Hz, 58 Hz and 150 Hz, 59 Hz and 150 Hz, 60 Hz and 150 Hz, 61 Hz and 150 Hz, 62 Hz and 150 Hz, 63 Hz and 150 Hz, 64 Hz and 150 Hz, 65 Hz and 150 Hz, 66 Hz and 150 Hz, 67 Hz and 150 Hz, 68 Hz and 150 Hz, 69 Hz and 150 Hz, 70 Hz and 150 Hz, 71 Hz and 150 Hz, 72 Hz and 150 Hz, 73 Hz and 150 Hz, 74 Hz and 150 Hz, 75 Hz and 150 Hz, 76 Hz and 150 Hz, 77 Hz and 150 Hz, 78 Hz and 150 Hz, 79 Hz and 150 Hz, 80 Hz and 150 Hz, 81 Hz and 150 Hz, 82 Hz and 150 Hz, 83 Hz and 150 Hz, 84 Hz and 150 Hz, 85 Hz and 150 Hz, 86 Hz and 150 Hz, 87 Hz and 150 Hz, 88 Hz and 150 Hz, 89 Hz and 150 Hz, 90 Hz and 150 Hz, 91 Hz and 150 Hz, 92 Hz and 150 Hz, 93 Hz and 150 Hz, 94 Hz and 150 Hz, 95 Hz and 150 Hz, 96 Hz and 150 Hz, 97 Hz and 150 Hz, 98 Hz and 150 Hz, 99 Hz and 150 Hz, 100 Hz and 150 Hz, 101 Hz and 150 Hz, 102 Hz and 150 Hz, 103 Hz and 150 Hz, 104 Hz and 150 Hz, 105 Hz and 150 Hz, 106 Hz and 150 Hz, 107 Hz and 150 Hz, 108 Hz and 150 Hz, 109 Hz and 150 Hz, 110 Hz and 150 Hz, 111 Hz and 150 Hz, 112 Hz and 150 Hz, 113 Hz and 150 Hz, 114 Hz and 150 Hz, 115 Hz and 150 Hz, 116 Hz and 150 Hz, 117 Hz and 150 Hz, 118 Hz and 150 Hz, 119 Hz and 150 Hz, 120 Hz and 150 Hz, 121 Hz and 150 Hz, 122 Hz and 150 Hz, 123 Hz and 150 Hz, 124 Hz and 150 Hz, 125 Hz and 150 Hz, 126 Hz and 150 Hz, 127 Hz and 150 Hz, 128 Hz and 150 Hz, 129 Hz and 150 Hz, 130 Hz and 150 Hz, 131 Hz and 150 Hz, 132 Hz and 150 Hz, 133 Hz and 150 Hz, 134 Hz and 150 Hz, 135 Hz and 150 Hz, 136 Hz and 150 Hz, 137 Hz and 150 Hz, 138 Hz and 150 Hz, 139 Hz and 150 Hz, 140 Hz and 150 Hz, 141 Hz and 150 Hz, 142 Hz and 150 Hz, 143 Hz and 150 Hz, 144 Hz and 150 Hz, 145 Hz and 150 Hz, 146 Hz and 150 Hz, 147 Hz and 150 Hz, 148 Hz and 150 Hz, 149 Hz and 150 Hz, 150 Hz and 150 Hz, 60 Hz and 100 Hz, 61 Hz and 100 Hz, 62 Hz and 100 Hz, 63 Hz and 100 Hz, 64 Hz and 100 Hz, 65 Hz and 100 Hz, 66 Hz and 100 Hz, 67 Hz and 100 Hz, 68 Hz and 100 Hz 69 Hz and 100 Hz, 70 Hz and 100 Hz, 60 Hz and 99 Hz, 61 Hz and 99 Hz, 62 Hz and 99 Hz, 63 Hz and 99 Hz, 64 Hz and 99 Hz, 65 Hz and 99 Hz, 66 Hz and 99 Hz 67 Hz and 99 Hz, 68 Hz and 99 Hz, 69 Hz and 99 Hz and 70 Hz and 99 Hz, and 61 Hz and 98 Hz, 62 Hz and 98 Hz, 63 Hz and 98 Hz, 64 Hz and 98 Hz, 65 Hz and 98 Hz, 66 Hz and 98 Hz, 67 Hz and 98 Hz, 68 Hz and 98 Hz, 69 Hz and 98 Hz and 70 Hz and 98 Hz.

In some embodiments, combinations of frequencies in an activation element or multiple activation elements leads to a Binaural beat. Such Binaural beats, or Binaural tones or Binaural frequencies, are apparent sounds caused by specific frequency combinations. Binaural beats may help induce relaxation, meditation, creativity, pain reduction, or other mental states. The effect on the brainwaves depends on the difference in frequencies of each tone, for example, if 300 Hz was played with one device and 310 Hz in the other, then the binaural beat would have frequency of 10 Hz. These slightly differing frequencies may also produce low-frequency pulsations in the amplitude and sound/frequency localization of a perceived sound/frequency when two tones at slightly different frequencies are presented separately. In the case of soundwaves, a beating tone may be perceived. The frequencies of the tones should be below 1,000 Hz for the beating to be noticeable and the difference between the two frequencies should be small (e.g., less than or equal to about 30 Hz) for the effect to occur.

Binaural beats may have health effects on mammals, although the exact health benefits possible are currently unverified. For instance, it is possible that Binaural beats simulate the effect of recreational drugs, help humans memorize and learn, help them stop smoking, help with dieting, help recover repressed memories, or improve athletic performance, among other things. The effect may be tied to the frequency range of the beat, as particular wave types are usually associated with different brain functions. For example, gamma waves in the range of approximately greater than 40 Hz are usually associated with higher mental activity, including perception, problem solving, fear, and consciousness. Beta waves in the range of approximately 13 to 39 Hz are usually associated with active, busy or anxious thinking and active concentration, arousal, cognition, and/or paranoia. Alpha waves in the range of approximately 7 to 13 Hz are usually associated with relaxation (while awake), pre-sleep and pre-wake drowsiness, REM sleep, dreams. Mu waves in the range of approximately 8 to 12 Hz are usually associated with sensorimotor rhythm. Theta waves in the range of approximately 4 to 7 Hz are usually associated with deep meditation/relaxation and NREM sleep. Delta waves in the range of approximately less than 4 Hz are usually associated with deep dreamless sleep or loss of body awareness. The precise boundaries between ranges vary among definitions, and there is no universally accepted standard.

Contemplated frequencies in accordance with the present disclosure, according to some embodiments are: from 65.4 Hz to 98 Hz on one activation element of an activation member and from 65.4 Hz to 98 Hz on another activation element of an activation member, where such frequencies result in a Binaural frequency of zero between the activation elements. Another pair of frequencies are from 65.4 Hz to 96 Hz on one activation element of an activation member and from 65.4 Hz to 98 Hz on another activation element of an activation member, where such frequencies result in a Binaural frequency of 1 to 2 Hz in between the activation elements. Another pair of frequencies are from 40 Hz to 98 Hz on one activation element of an activation member and from 40 Hz to 98 Hz on another activation element of an activation member, where such frequencies result in a Binaural frequency of zero between the activation elements. Another pair of frequencies are from 40 Hz to 80 Hz on one activation element of an activation member and from 40 Hz to 80 Hz on another activation element of an activation member, where such frequencies result in a Binaural frequency of zero between the activation elements And lastly, another pair of frequencies are from 40 Hz to 79 Hz on one activation element of an activation member and from 41 Hz to 81 Hz on another activation element of an activation member, where such frequencies result in a Binaural frequency of between 1 and 2 Hz between the activation elements.

Further still, in accordance with the present disclosure, according to some embodiments, the amplitude of the signal can be adjusted to adjust the sound pressure generated by activation member. It is contemplated that the amplitude may be doubled or increased even more to deliver the therapy in accordance with the present disclosure, according to some embodiments. In accordance with the disclosure, the activation member may be configured to provide a sound pressure between: 0 to 150 decibels, 0 to 100 decibels, 0 to 99 decibels, 0 to 98 decibels, 0 to 97 decibels, 0 to 96 decibels, 0 to 95 decibels, 0 to 94 decibels, 0 to 93 decibels, 0 to 92 decibels, 0 to 91 decibels, 0 to 90 decibels, 0 to 89 decibels, 0 to 88 decibels, 0 to 87 decibels, 0 to 86 decibels, 0 to 85 decibels, 0 to 84 decibels, 0 to 83 decibels, 0 to 82 decibels, 0 to 81 decibels, 0 to 80 decibels, 0 to 79 decibels, 0 to 78 decibels, 0 to 77 decibels, 0 to 76 decibels, 0 to 75 decibels, 0 to 74 decibels, 0 to 73 decibels, 0 to 72 decibels, 0 to 71 decibels, 0 to 70 decibels, 0 to 69 decibels, 0 to 68 decibels, 0 to 67 decibels, 0 to 66 decibels, 0 to 65 decibels, 0 to 64 decibels, 0 to 63 decibels, 0 to 62 decibels, 0 to 61 decibels, 0 to 60 decibels, 0 to 59 decibels, 0 to 58 decibels, 0 to 57 decibels, 0 to 56 decibels, 0 to 55 decibels, 0 to 54 decibels, 0 to 53 decibels, 0 to 52 decibels, 0 to 51 decibels, 0 to 50 decibels, 0 to 49 decibels, 0 to 48 decibels, 0 to 47 decibels, 0 to 46 decibels, 0 to 45 decibels, 0 to 44 decibels, 0 to 43 decibels, 0 to 42 decibels, 0 to 41 decibels, 0 to 40 decibels, 0 to 39 decibels, 0 to 38 decibels, 0 to 37 decibels, 0 to 36 decibels, 0 to 35 decibels, 0 to 34 decibels, 0 to 33 decibels, 0 to 32 decibels, 0 to 31 decibels, 0 to 30 decibels, 0 to 29 decibels, 0 to 28 decibels, 0 to 27 decibels, 0 to 26 decibels, 0 to 25 decibels, 0 to 24 decibels, 0 to 23 decibels, 0 to 22 decibels, 0 to 21 decibels, 0 to 20 decibels, 0 to 19 decibels, 0 to 18 decibels, 0 to 17 decibels, 0 to 16 decibels, 0 to 15 decibels, 0 to 14 decibels, 0 to 13 decibels, 0 to 12 decibels, 0 to 11 decibels, 0 to 10 decibels, 0 to 9 decibels, 0 to 8 decibels, 0 to 7 decibels, 0 to 6 decibels, 0 to 5 decibels, 0 to 4 decibels, 0 to 3 decibels, 0 to 2 decibels, 0 to 1 decibels, 0 to 0.5 decibels, 0 to 0.25 decibels, 10 to 100 decibels, 20 to 100 decibels, 30 to 100 decibels, 40 to 100 decibels, 50 to 100 decibels, 60 to 100 decibels, 70 to 100 decibels, 80 to 100 decibels, 90 to 100 decibels, 10 to 75 decibels, 20 to 75 decibels, 30 to 75 decibels, 40 to 75 decibels, 50 to 75 decibels, 60 to 75 decibels, 70 to 75 decibels, 10 to 65 decibels, 20 to 65 decibels, 30 to 65 decibels, 40 to 65 decibels, 50 to 65 decibels and 60 to 65 decibels, 20 to 30 decibels, 30 to 40 decibels, 40 to 50 decibels, 50 to 60 decibels, 60 to 70 decibels, 70 to 75 decibels, 80 to 90 decibels, 50 to 75 decibels and 50 to 65 decibels.

In accordance with the present disclosure, according to some embodiments, it is contemplated that the activation member may be activated for a time period between about 1 second and 24 hours. In other embodiments, the activation member may be activated for a time period of between about 1 second and 12 hours, 1 second and 11 hours, 1 second and 10 hours, 1 second and 9 hours, 1 second and 8 hours, 1 second and 7 hours, 1 second and 6 hours, 1 second and 5 hours, 1 second and 4 hours, 1 second and 3 hours 1 second and 2 hours, and 1 second and 1 hour, 1 second and 45 minutes, 1 second and 30 minutes, 1 second and 20 minutes, 1 second and 15 minutes, 1 second and 10 minutes, 1 second and 5 minutes and 1 second and 1 minute.

The overall process may be conducted for a time period between 1 second and 24 hours, 1 second and 23 hours, 1 second and 22 hours, 1 second and 21 hours, 1 second and 20 hours, 1 second and 19 hours, 1 second and 18 hours, 1 second and 17 hours, 1 second and 16 hours, 1 second and 15 hours, 1 second and 15 hours, 1 second and 14 hours, 1 second and 13 hours, 1 second and 12 hours, 1 second and 11 hours, 1 second and 10 hours, 1 second and 9 hours, 1 second and 8 hours, 1 second and 7 hours, 1 second and 6 hours, 1 second and 5 hours, 1 second and 4 hours, 1 second and 3 hours, 1 second and 2 hours, 1 second and 1 hour, 1 second and 45 minutes, 1 second and 30 minutes, 1 second and 15 minutes, 1 second and 10 minutes, 1 second and 5 minutes, 1 second and 1 minute.

In accordance with the present disclosure, according to some embodiments, the catheter may be factory programmed to utilize a certain frequency or range of frequencies to measure nerve activity. Alternatively, the frequencies may be selected and programmed or chosen from memory by a health care provider based upon the detection of a patient's nerves in response to a specific frequency or range of frequencies.

It is further contemplated in accordance with the present disclosure, according to some embodiments, that a catheter may additionally be connected to a processor and/or computing device to receive and/or compute and/or analyze the measured signals. In some embodiments, a computing device may be additionally in communication with other sensors, such as a blood pressure monitor, heart rate monitor, pulse oximetry monitor, electrocardiogram (EKG/ECG), or glucose sensor.

It is further contemplated that any of the above sensors could be incorporated into the catheter device in accordance with the present disclosure, according to some embodiments. If incorporated into the catheter, the data from each of the additional sensors could be utilized by the program to alter activation/measurement signals of the catheter based upon data received from the various sensors.

In yet another embodiment, a catheter can also be utilized to identify the location of nerves within a lumen, whereby the vibrational energy is utilized to activate the nerves and the sensors are utilized to detect nerve activity, when a high signal to noise ratio is detected between the activation member and the measurement member, this informs the user of an area in which denervation should be performed. Therefore, the catheter can be utilized to identify nerves for denervation, thereby potentially leading to possible better patient outcomes.

It shall be understood that although the various catheter embodiments of the present disclosure shown and describe two separate expandable members, it shall be understood that a single expandable member can be utilized in order to perform the methods disclosed herein. For example, using a single expandable member, the measurement sensors can be disposed onto the single expandable member which is also the activation member as well. Alternatively, an activation member on a catheter may send energy to the nerves while an alternative measurement means is used to measure the nerve activity. Also alternatively, a measurement member on a catheter may measure nerve activity after the nerves have been activated by an alternative means.

It is further contemplated that in addition to the embodiments disclosed herein, an HCP may also use other means in either in conjunction, before, or after to determine whether there is still nerve activity. For instance, an HCP may employ a functional MRI, PET scan, or measure kidney function via serum or urine electrolyte, such as sodium or potassium levels, BUN or creatinine levels, glomerular filtration rate, renin, angiotensinogen, angiotensin I, angiotensin II, ACE, aldosterone, epinephrine, norepinephrine, dopamine, metanephrine, vanillylmandelic acid, cortisol, 5-HIAA, or other levels, such as levels indicative of sympathetic nerve function, or other means (such as blood pressure) to assess nerve response.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the disclosure should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “positioning a device for producing vibrational energy inside a lumen” include “instructing the positioning of a device for producing vibrational energy inside a lumen.” The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers, and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

What is claimed is:

1. A device for producing vibrational energy, comprising
an elongated shaft member, the elongated shaft including a proximal end and a distal end;
an activation member associated with the elongated shaft member, wherein the activation member includes a vibration source; and
a measurement member associated with the elongated shaft member.

2. The device of claim 1, wherein the measurement member includes a sensor.

3. The device of claim 2, wherein the sensor is an electrode.

4. The device of claim 1, wherein the activation member comprises an expandable balloon.

5. The device of claim 1, wherein the activation member comprises a nitinol cage.

6. The device of claim 4, wherein the activation member further comprises a rotatable member within the expandable balloon.

7. The device of claim 6, wherein the rotation source strikes a tuned arm to creating vibratory energy.

8. The device of claim 6, wherein the rotation source strikes struts positioned on the expandable balloon, thereby creating vibratory energy.

9. The device of claim 7, wherein the rotation source creates a vibration by rotating, and wherein the rotation source may be advanced and retracted to change the amplitude of vibrations within the expandable balloon.

10. The device of claim 2, wherein the vibration source is selected from the group comprising a piezo speaker, a haptic speaker, an ultrasonic speaker, an ultrasonic transducer, or an electroactive polymer.

11. The device of claim 2, wherein the vibration source comprises two piezo speakers.

12. The device of claim 11, wherein, the two piezo speakers create two frequencies that create a binaural beat.

13. The device of claim 1, wherein the vibration energy comprises a first frequency between about 25 Hz and about 150 Hz.

14. The device of claim 1, wherein the vibration energy comprises a second frequency between about 25 Hz and about 150 Hz.

15. The device of claim 1, wherein the activation member comprises a fluid channel configured to generate turbulence, thereby releasing vibratory energy.

16. The device of claim 1, further including an ablation member configured to ablate the nerve of interest via a non-vibratory energy modality.

17. A method of detecting nerve activity, comprising
inserting a catheter into the lumen of a mammal having a sidewall, wherein the catheter comprises an activation member, and further comprising a measurement member spaced apart on the catheter from the activation member, the measurement member configured to detect energy from a nerve of interest proximate or within the sidewall of the lumen;
delivering energy from the activation member toward the nerve of interest adjacent to the lumen where the catheter is placed, wherein the activation member produces vibratory energy to activate the nerves; and
detecting nerve activity using the measurement member when the measurement member measures a signal greater than 0.

18. The method of claim 16, wherein the lumen is a renal artery, and the nerve of interest is a renal sympathetic nerve.

19. The method of claim 16, wherein the vibratory energy comprises a binaural beat.

20. The method of claim 17, further comprising the step of ablating the nerve of interest.
